(19) United States
(12) Patent Application Publication
(10) Pub. No.: US 2007/0249673 A1
(43) Pub. Date: Oct. 25, 2007

(54) Method for administering tolperisone

(76) Inventors: Angelika Bodenteich, Wien (AT); Josef Bockmann, Modling (AT); Werner J. Frantsits, Wien (AT)

Correspondence Address: ROBINS & PASTERNAK LLP, 1731 EMBARCADERO ROAD, SUITE 230, PALO ALTO, CA, 94303, US

(21) Appl. No.: 11/788,754

(22) Filed: Apr. 20, 2007

Related U.S. Application Data
(60) Provisional application No. 60/794,149, filed on Apr. 20, 2006.

(51) Int. Cl. A61K 31/445; A61K 9/48; A61K 9/20

(52) U.S. Cl. 514/317; 424/451; 424/464

(57) ABSTRACT

The present invention is directed to methods of administering tolperisone (2,4′-dimethyl-3-piperidinopropiophenone; 1-propanone, 2-methyl-1-(4-methylphenyl)-3-(-piperidinyl)), and kits comprising the same.

### CROSS REFERENCE TO RELATED APPLICATIONS

This application claims the benefit of U.S. provisional application Ser. No. 60/794,149, filed Apr. 20, 2006, from which application priority is claimed under 35 USC §119(e)(1) and which application is incorporated herein by reference in its entirety.

### FIELD OF THE INVENTION

The present invention relates generally, in one or more embodiments, to methods of administering tolperisone (2,4′-dimethyl-3-piperidinopropiophenone; 1-propanone, 2-methyl-1-(4-methylphenyl)-3-(-piperidinyl)), and compositions and kits comprising the same.

### BACKGROUND OF THE INVENTION

Tolperisone, also referred to as (R,S)2,4′-dimethyl-3-piperidinopropiophenone, is a centrally-acting muscle relaxant that has been used for the symptomatic treatment of spasticity and muscle spasm (Martindale, The Extra Pharmacopoeia, 30^{th ed}., p. 1211). Tolperisone has also been used in the treatment of conditions which include dysmenorrhea, climacteric complaints, lockjaw, and neurolatyrism.

The chemical structure of tolperisone is shown below. 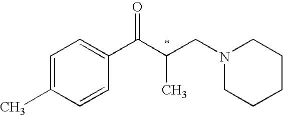
As can be seen by the foregoing structure, tolperisone contains a chiral center (as indicated by the asterisk). Racemic tolperisone is commercially available as the hydrochloride salt and sold under trade names such as Mydeton®, Mydocalm®, Midocalm® and Muscalm®. The chiral separation of tolperisone into its R(−) and S(+) enantiomers has also been described (See JP-A-53-40779).

Tolperisone has been shown to exhibit membrane-stabilizing effects in the central and peripheral nervous system (Ono, H., et al., J. Pharmacobio. Dynam. 1984, 7, 171-178). Tolperisone and its salts are used for improving not only different symptoms related to spastic paralysis, but also for improving muscle tone which originates from diseases or conditions such as cervical syndrome, inflammation of the joints, and back pain. Recently, the use of tolperisone for treating neuropathic pain and pain associated with various nervous system disorders has also been suggested (U.S. Patent Application No. 2006/0004050).

Despite its proven pharmacological efficacy, oral administration of tolperisone is problematic, since it is rapidly metabolized and cleared from the body. To achieve a therapeutic dosage upon administration, patients must take several oral dosages of tolperisone daily, which can present problems with patient compliance, and potentially cause damage to the gastro-intestinal tract.

Several approaches have been described to ameliorate the drawbacks associated with oral administration of tolperisone due to its short circulating half-life. For example, U.S. Pat. No. 6,500,455 describes various controlled release pharmaceutical preparations of tolperisone, e.g., hydrogel-based formulations, coated tablets, and microcapsules. U.S. Patent Application No. 2005/0196451 describes controlled release formulations of tolperisone in which tolperisone is combined with a methacrylate-based polymer such as Eudragit®. Additional approaches for overcoming the rapid in-vivo metabolism of orally administered tolperisone include the administration of particular enantiomeric ratios of tolperisone as described in WO 00/59508. However, additional approaches for improving the pharmacokinetic profile of tolperisone are needed, in particular, approaches that are straightforward, and don't require multiple and complex separation or formulation steps.

It is believed that the present invention meets those needs.

### SUMMARY OF THE INVENTION

The present invention relates to methods for administering tolperisone. In investigating the administration of tolperisone, the inventors have discovered that the administration of tolperisone to a mammalian (e.g., human) subject in the fed state (versus the fasted state) has an advantageous effect on the pharmacokinetics of the drug. Indeed, the inventors have discovered that the oral administration of tolperisone to a subject in the fed state is effective to (i) increase the bioavailability of tolperisone, as well as (ii) delay its absorption, in comparison to the conventional approach of oral administration of tolperisone to a subject in the fasted state.

Thus, in one aspect, the invention is directed to, in one or more general embodiments, a method of administering a therapeutically effective dosage of tolperisone to a subject suffering from a condition responsive to treatment with tolperisone, wherein the subject is in a fed state, i.e., wherein the administering takes place within approximately one hour following the commencement of meal consumption by the subject, preferably within approximately 30 minutes following the commencement of meal consumption.

In yet another aspect, the invention provides a method for administering tolperisone, wherein a therapeutically effective dosage of tolperisone is administered to a subject suffering from a condition responsive to treatment with tolperisone, and the subject is in a fed state. The administering is effective to achieve an elimination half-life of tolperisone in plasma that is prolonged over that achieved upon administration of tolperisone to the subject in a fasted state.

In one or more particular embodiments, the method is effective to achieve an elimination half-life of tolperisone that is prolonged by at least 10%, preferably 20%, or even more preferably 30% or greater, over that achieved upon administration of tolperisone to the subject in a fasted state.

In yet another aspect, a method for administering tolperisone is provided, wherein a therapeutically effective dosage of tolperisone is administered to a subject suffering from a condition responsive to treatment with tolperisone and the subject is in a fed state, to thereby increase the bioavailability of tolperisone over that achieved upon administration of tolperisone to the subject in a fasted state.

In one or more particular embodiments, the method is effective to increase the bioavailability of tolperisone by at least 10% over that achieved by administration of tolperisone to a subject in a fasted state, preferably by at least 20%, and even more preferably by 30% or greater than that achieved upon administration of tolperisone to a subject in a fasted state.

Mammalian subjects suitable for treatment using the methods of the invention include those suffering from one or more of the following conditions: spasticity, muscle spasm, dysmenorrhea, climacteric complaints, lockjaw, neurolatyrism, deteriorated muscle tone originating from diseases or conditions such as cervical syndrome, inflammation of the joints, and back pain, neuropathic pain, and pain associated with various nervous system disorders.

Tolperisone for use in the invention may be a racemate, a chiral mixture (a mixture of enantiomers), or a pure (R) or (S)— enantiomer; additionally, tolperisone may be used in any of its pharmaceutically acceptable salt forms.

According to any one or more particular embodiments of the method of the invention, tolperisone is administered at a therapeutically effective dosage ranging from 10-3000 milligrams daily, preferably at a therapeutically effective dosage ranging from approximately 50 milligrams to 1800 milligrams daily. Such therapeutically effective amount may be administered as a single dose daily, or as several doses over the course of a day. Preferably, tolperisone is administered at a therapeutically effective dosage ranging from approximately 75 milligrams to 1500 milligrams daily.

In one or more preferred embodiments, tolperisone is administered orally.

In yet another aspect of the invention, provided is a kit comprising tolperisone in packaged form, and instructions for administering tolperisone within approximately one hour of eating.

In a particular embodiment of the kit, tolperisone is provided in a dosage form selected from the group consisting of a tablet, syrup, suspension, and capsule.

In yet another embodiment, tolperisone is provided in unit dosage form.

The therapeutic dosage amount may be achieved by administration once daily (i.e., in a single dose), twice daily (i.e., in two separate doses), three times daily, or may be administered as multiple doses, over a time course of several days, weeks, or even months.

Each of the herein-described features of the invention is meant to apply equally to each and every embodiment as described herein, unless otherwise indicated.

Additional objects, advantages and novel features of the invention will be set forth in the description that follows, and in part, will become apparent to those skilled in the art upon reading the following, or may be learned by practice of the invention.

### BRIEF DESCRIPTION OF THE FIGURES

FIG. 1 is a plot of mean plasma concentrations of tolperisone in various treatment groups following administration in a single dose 4-way crossover study as described in detail in the Example.

FIG. 2 is a plot of mean plasma concentrations of 4-HM-tolperisone (4-hydroxymethyl-tolperisone) in various treatment groups following administration in a single dose 4-way crossover study as described in detail in the Example.

### DETAILED DESCRIPTION OF THE INVENTION

The practice of the present invention will employ, unless otherwise indicated, conventional methods of chemistry, biochemistry, and pharmacology, within the skill of the art. Such techniques are explained fully in the literature. See, e.g.; A. L. Lehninger, Biochemistry (Worth Publishers, Inc., current addition); Morrison and Boyd, Organic Chemistry (Allyn and Bacon, Inc., current addition); J. March, Advanced Organic Chemistry (McGraw Hill, current addition); Remington: The Science and Practice of Pharmacy, A. Gennaro, Ed., 20^{th} Ed.; Goodman & Gilman The Pharmacological Basis of Therapeutics, J. Griffith Hardman, L. L. Limbird, A. Gilman, 10^{th} Ed; Handbook of Pharmaceutical Manufacturing Formulations, S. K. Niazi (ed.), CRC Press, 2004; Basic and Clinical Pharmacology, 18^{th} Edition, Katzung, B. G. (ed.), Appleton & Lange, Norwalk, Conn., 2001.

All publications, patents and patent applications cited herein, whether supra or infra, are hereby incorporated by reference in their entirety.

Definitions

Before describing the present invention in detail, it is to be understood that this invention is not limited to particular administration modes, patient populations, and the like, as such may vary, as will be apparent from the accompanying description and figures.

It must be noted that, as used in this specification and the intended claims, the singular forms “a,” “an,” and “the” include plural referents unless the context clearly dictates otherwise. Thus, for example, reference to “a drug” includes a single drug as well as two or more of the same or different drugs, reference to “an optional excipient” refers to a single optional excipient as well as two or more of the same or different optional excipients, and the like.

In describing and claiming the present invention, the following terminology will be used in accordance with the definitions described below.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Active molecule” or “active agent” as described herein includes any agent, drug, compound, composition of matter or mixture which provides some pharmacologic, often beneficial, effect that can be demonstrated in-vivo or in vitro. This includes foods, food supplements, nutrients, nutriceuticals, drugs, vaccines, antibodies, vitamins, and other beneficial agents. As used herein, the terms further include any physiologically or pharmacologically active substance that produces a localized or systemic effect in a patient.

“Substantially” or “essentially” means nearly totally or completely, for instance, 95% or greater of some given quantity.

“Optional” or “optionally” means that the subsequently described circumstance may or may not occur, so that the description includes instances where the circumstance occurs and instances where it does not.

The terms “subject”, “individual” or “patient” are used interchangeably herein and refer to a vertebrate, preferably a mammal. Mammals include, but are not limited to, murines, rodents, simians, humans, farm animals, sport animals and pets.

The terms “pharmacologically effective amount” or “therapeutically effective amount” of a composition or agent such as tolperisone, refer to a nontoxic but sufficient amount of the composition or agent to provide the desired response. The exact amount required will vary from subject to subject, depending on the species, age, and general condition of the subject, the severity of the condition being treated, the particular drug form or combination of drugs employed, mode of administration, and the like. An appropriate “effective” amount in any individual case may be determined by one of ordinary skill in the art using routine experimentation, based upon the information provided herein.

The terms “about” and “approximately”, particularly in reference to a given quantity, is meant to encompass deviations of plus or minus five percent.

“Treatment” or “treating” a particular condition refers to alleviation of symptoms of the condition in question, as well as elimination of the condition in question. For example, “treatment” or “treating” pain such as neuropathic pain includes: (1) preventing pain, i.e. causing pain not to develop or to occur with less intensity in a subject that may be exposed to or predisposed to pain but does not yet experience or display pain, (2) inhibiting pain, i.e., arresting the development or reversing pain, or (3) relieving pain, i.e., decreasing the amount of pain experienced by the subject.

Methods of Administration

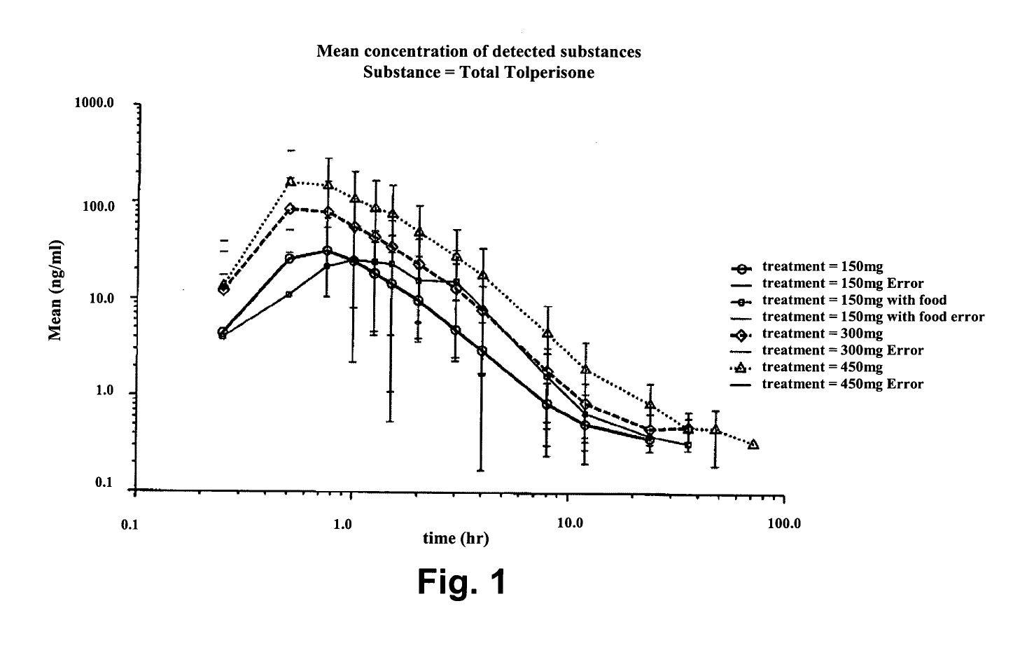

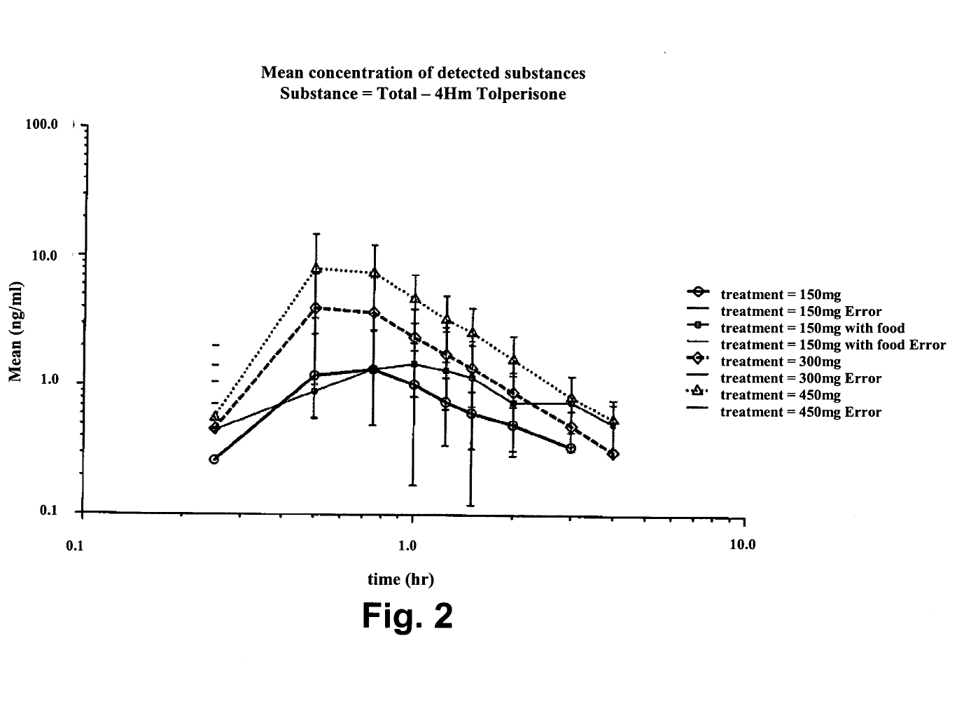

As set forth above, the present invention is directed, in one or more embodiments, to a method of administering tolperisone. In particular, the invention is directed to a method of orally administering tolperisone to a subject in a fed (i.e., non-fasted) state. As can be seen from the accompanying Example, for the subject group in which administration of tolperisone was accompanied by a meal, an enhancement in both bioavailability and absorption was observed when compared to the fasted subject groups. See, e.g., FIGS. 1 and 2.

Conditions Responsive to Treatment with Tolperisone

Tolperisone is a centrally-acting muscle relaxant that acts on the central nervous system and is used mainly for the treatment of elevated muscle tone and tension, as well as for certain circulatory problems in the extremities. Tolperisone has been found to reduce experimental hypertonia and decerabration rigidity, as well as inhibit reticulospinal reflex facilitation without affecting cortical functions. It also improves peripheral blood flow (Toperin® Package Insert). Thus, tolperisone is useful in treating a number of conditions. For example, tolperisone may be administered to a subject suffering from one of more of the following conditions, which include: muscle spasm, spastic syndromes, muscle soreness, myotonia, dysmenorrhea, climacteric complaints, lockjaw, neurolatyrism, osteoarthritis or rheumatoid arthritis (when administered in combination with a non-steroidal anti-inflammatory drug), rheumatic diseases, fibromyalgia syndrome, occupational and sport-related stress, spasticity caused by neurological diseases, multiple sclerosis, myelopathy, encephalomyelitis, muscular hypertension, muscular contracture, spinal automatism, obliterative vascular diseases (e.g., obliterative arteriosclerosis, diabetic angiopathy, obliterative thromboangitis, Raynaud's disease, diffuse scleroderma), disorders due to injured innervation of the vessels (acrocyanosis, intermittent angioneurotic dysbasis), neuropathic pain, and in individual cases, post-thrombotic venous and lymphatic circulation disorders, and crural ulcer (Myolax® Package insert).

Patient Population

Subjects to whom tolperisone may be administered in accordance with the invention include both children (aged three months to 18 years), and adults (18 years and older).

Routes of Administration

Methods of administering therapeutic formulations of tolperisone include but are not limited to oral, intra-arterial, intrathecal, intraspinal, intramuscular, intraperitoneal, intravenous, intranasal, and inhalation routes. Preferred routes of administration are intramuscular, intravenous, and oral. In a particularly preferred embodiment, tolperisone is administered orally. However, tolperisone may be administered by any suitable route, including without limitation, oral, rectal, nasal, topical (including transdermal, aerosol, buccal and sublingual), vaginal, parenteral (including subcutaneous, intramuscular, intravenous and intradermal), intrathecal, and pulmonary. The preferred route will, of course, vary with the condition and age of the recipient, the particular condition being treated, and the specific combination of drugs employed, if any.

Oral dosage forms include tablets, lozenges, capsules, syrups, oral suspensions, emulsions, granules, and pellets. Commercially available oral dosage forms of tolperisone include film-coated tablets (Toperin®, Myolax®, Tolcalm®, Midocalm®). Alternative formulations include aerosols, transdermal patches, gels, creams, ointments, suppositories, powders or lyophilates that can be reconstituted, as well as liquids. Examples of suitable diluents for reconstituting solid compositions, e.g., prior to injection, include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned.

A pharmaceutical composition of tolperisone for topical administration may be formulated as an ointment, cream, suspension, lotion, powder, solution, paste, gel, spray, aerosol or oil.

Alternatively, the formulation may be in the form of a patch (e.g., a transdermal patch) or a dressing such as a bandage or adhesive plaster impregnated with active ingredients and optionally one or more excipients or diluents. Topical formulations may additionally include a compound that enhances absorption or penetration of the ingredients through the skin or other affected areas, such as dimethylsulfoxidem bisabolol, oleic acid, isopropyl myristate, and D-limonene, to name a few.

Formulations suitable for parenteral administration include aqueous and non-aqueous isotonic sterile solutions suitable for injection, as well as aqueous and non-aqueous sterile suspensions. A formulation of tolperisone for either intramuscular or intravenous injection, Crenol®, is commercially available from Woonam Pharm. Co., Ltd. Parenteral formulations are optionally contained in unit-dose or multi-dose sealed containers, for example, ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the types previously described.

A tolperisone formulation of the invention may also be in the form of a sustained release formulation, such that each of the drug components is released or absorbed slowly over time, when compared to a non-sustained release formulation. Sustained release formulations may employ pro-drug forms of the active agent, delayed-release drug delivery systems such as liposomes or polymer matrices, hydrogels, or covalent attachment of a polymer such as polyethylene glycol to the active agent. Illustrative sustained release formulations of tolperisone are described in U.S. Patent Application Publication No. 2005/0196451.

In addition to the ingredients particularly mentioned above, the formulations of the invention may optionally include other agents conventional in the pharmaceutical arts and particular type of formulation being employed, for example, for oral administration forms, the composition for oral administration may also include additional agents as sweeteners, thickeners or flavoring agents. These foregoing pharmaceutical excipients along with other excipients are described in “Remington: The Science & Practice of Pharmacy”, 19^{th} ed., Williams & Williams, (1995), the “Physician's Desk Reference”, 52^{nd ed}., Medical Economics, Montvale, N.J. (1998), and Kibbe, A. H., Handbook of Pharmaceutical Excipients, 3^{rd} Edition, American Pharmaceutical Association, Washington, D.C., 2000.

The method of the present invention is also useful in veterinary applications.

Dosage Amount

Therapeutic amounts of tolperisone can be empirically determined and will vary with the particular condition being treated, the subject, and the like. The actual dose to be administered will vary depending upon the age, weight, and general condition of the subject as well as the severity of the condition being treated, the judgment of the health care professional, and particular dosage form being administered.

Therapeutically effective amounts can be determined by those skilled in the art, and will be adjusted to the requirements of each particular case. Generally, a therapeutically effective amount of tolperisone for an adult will range from a total daily dosage of between about 10 and 3000 mg/day, preferably, in an amount between 25-2000 mg/day, more preferably, in an amount between about 50-1800 mg/day. Typical dosage ranges for adults include total daily dosage ranges from about 150-1000 mg/day, preferably from about 150 to about 750 mg/day, administered as either a single dosage or as multiple dosages. Preferred in certain embodiments are divided dosages over the course of a day, e.g., a recommended daily dose divided into five doses, or four doses, or three doses, or two doses. Preferred dosage amounts include dosages from about 50 mg to 450 mg twice daily or three times daily. That is to say, dosage amounts may be selected from 50 mg/day, 100 mg/day, 150 mg/day, 200 mg/day, 250 mg/day, 300 mg/day, 350 mg/day, 400 mg/day, 450 mg/day, 500 mg/day or more. Depending upon the dosage amount and precise condition to be treated, administration can be one, two, or three times daily for a time course of one day to several days, weeks, months, and even years, and may even be for the life of the patient. Illustrative dosing regimes will last a period of at least about a day, a week, from about 1-4 weeks, from 1-3 months, from 1-6 months, from 1-50 weeks, from 1-12 months, or longer. Dosage amounts for children ranging in age from 3 months to 18 years in age range from about 1-25 mg/kg/day, preferably from about 2-15 mg/day, in from about 2-4 divided doses, preferably 3 doses. Exemplary recommended dosage ranges for children include 5-10 mg/kg/day and from 2-4 mg/kg/day, in 2-3 divided doses.

Practically speaking, a unit dose of any given composition of the invention or active agent can be administered in a variety of dosing schedules, depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, every other day, three times weekly, twice weekly, once weekly, twice monthly, once monthly, and so forth.

Administering with Meals

As can be seen from the Example, the inventors have discovered a significantly beneficial effect in administering tolperisone at mealtime. Specifically, an increase in both the bioavailability and elimination half-life of tolperisone can be achieved by administering tolperisone to a subject who is in a fed state versus one who is in a fasted state.

Thus, in accordance with one aspect of the invention, tolperisone in any form suitable for administration to a mammal is administered to a subject who is in a non-fasted state. That is to say, tolperisone is administered to a subject along with a meal. More particularly, tolperisone is administered to a subject who is concurrently consuming a meal (i.e., is taken with a meal or at mealtime), or who has begun meal consumption within about one hour prior to administration of tolperisone. Preferably, administration of tolperisone is within about 30 minutes of commencement of meal consumption by the subject. By “meal” is meant any food article containing at least about 200 food calories (one food calorie equals 4.187 joules). Preferably, tolperisone is administered as described above with a meal containing from about 200-1200 or more calories. Preferably, the meal is one having a fat content of at least about 15% fat, more preferably is one having a fat content of at least about 20% fat. In one or more embodiments of the method, the meal contains an amount of fat selected from the group consisting of at least about 25% fat, at least about 30% fat, at least about 40% fat, at least about 50% fat, or at least about 55% fat. In one or more embodiments, the subject has completed meal consumption prior to administration of tolperisone. That is to say, in certain embodiments, a subject has just completed a meal as described above prior to administering tolperisone. In yet additional embodiments, a subject has completed a meal as described above within about 5 minutes, or 10 minutes, or 15 minutes or 20 minutes or even 30 minutes, prior to administering tolperisone.

The method of the invention is effective to achieve an elimination half-life of tolperisone that is prolonged by at least about 5%, preferably by at least 10%, or even more preferably by at least 20% or even 30% or greater than that achieved upon administration of tolperisone to the subject in a fasted state. For the purposes of the present invention, a subject that is in a fasted state is one who has consumed no food for at least about two hours prior to administration of tolperisone, preferably who has consumed no food for at least three hours prior to administration of tolperisone. In instances in which tolperisone is administered only once daily, a subject in a fasted state may be one who has consumed no food for at least 6 hours prior to administration of tolperisone.

In yet another embodiment, the method of the invention is effective to achieve a bioavailability of tolperisone that is improved over the bioavailability of tolperisone when administered to the subject in a fasted state. Ideally, the bioavailability of tolperisone is enhanced or increased by at least about 10%, preferably by at least about 20%, and even more preferably by at least about 30% or more over that upon administration of tolperisone to the subject in a fasted state.

Forms of Tolperisone

Tolperisone, as referred to herein, is meant to include any and all pharmaceutically acceptable salt forms thereof, prodrug forms (e.g., the corresponding ketal), solvates, and the like, as appropriate for use in its intended formulation for administration. In many of its commercially available forms, tolperisone as provided as the hydrochloride salt.

Tolperisone includes a chiral center. Thus, the term “tolperisone” as used herein is meant to encompass, where applicable, any and all enantiomers, mixtures of enantiomers including racemic mixtures and non-racemic mixtures (see, e.g., U.S. Pat. No. 6,500,455), prodrugs, pharmaceutically acceptable salt forms, hydrates (e.g., monohydrates, dihydrates, etc.), solvates, different physical forms (e.g., crystalline solids, amorphous solids), metabolites, and the like.

Racemic tolperisone (+) is commercially available. Its preparation has also been described. The enantiomers of tolperisone, (+)-tolperisone, and (−)-tolperisone, can be obtained using chiral separation methods, e.g., chiral HPLC, using columns such as CHIRAL-AGP (Chrom Tech, Ltd., Cheshire, UK) or Whelk 0 1 (Regis Technologies, Morton Grove, Ill.), or by capillary electrophoresis (Mutsunaga, H., et al., Electrophoresis, 2003, Aug. 24(5), 2442-7.

Kits

Also provided herein is a kit comprising tolperisone, in packaged form, accompanied by instructions for use. For example, the kit includes instructions for administering a recommended dosage of tolperisone at meal time, where meal time is as described above. For example, the kit comprises tolperisone in unit dosage form, along with instructions for use. Tolperisone may be packaged in any manner suitable for administration, so long as the packaging, when considered along with the instructions for administration, clearly indicates the manner in which the drug component is to be administered. For example, when tolperisone is in the form of a coated tablet, then the kit may comprise a sealed container of coated tablets, blister strips containing the tablets, or the like. The packaging may be in any form commonly employed for the packaging of pharmaceuticals, and may utilize any of a number of features such as different colors, wrapping, tamper-resistant packaging, blister packs or strips, dessicants, and the like.

It is to be understood that while the invention has been described in conjunction with preferred specific embodiments, the foregoing description as well as the examples that follow are intended to illustrate and not limit the scope of the invention. Other aspects, advantages and modifications within the scope of the invention will be apparent to those skilled in the art to which the invention pertains.

### EXAMPLES

In the following examples, efforts have been made to ensure accuracy with respect to numbers used (e.g., amounts, temperatures, etc.) but some experimental error and deviation should be accounted for. Each of the following examples is considered to be instructive to one of ordinary skill in the art for carrying out one or more of the embodiments described herein.

### Example 1

### Food Effect of Tolperisone in Healthy Male Volunteers

One objective of the study was to explore the food effect following single oral doses of tolperisone.

Overall Study Design. The study was designed as an open-label, single dose, 4-way crossover study in 24 healthy male subjects. Randomization was performed according to the Latin Square Block design. Six subjects each were distributed to the four treatment arms. Each subject completed the study having received a single dose of 150 mg, 300 mg, and 450 mg of tolperisone once daily in a fasted state, and 150 mg of tolperisone with food. The wash-out period between study periods was at least seven days.

Subjects. 24 healthy male subjects were planned to participate in the study. Two subjects withdrew from treatment on study day 2 of the second treatment sequence. These two subjects were replaced, so that a total of 24 subjects completed the study. The study group was composed of healthy male Caucasians, aged between 18-50 years, with a body mass index of 19-28 kg/m².

Active Substance. Tolperisone, 2-methyl-1-(4-methylphenyl)-3-(1-piperidinyl)-1-propanone, was supplied in coated tablets of 150 mg (“SPH-3047”, Sanochemia Pharmazeutica AG).

Dosage Regimen. The dosage regimen was a four-way crossover. Within four treatment sequences, each subject received each of the following doses once.

| | |
|---|---|
| Period 1: | 150 mg - fasting (1 tablet) |
| Period 2: | 300 mg - fasting (2 tablets) |
| Period 3: | 450 mg - fasting (3 tablets) |
| Period 4: | 150 mg - with food (1 tablet) |

Duration. The duration of each treatment sequence was 4 days, in which each subject received one single dose of study medication. Each subject underwent four treatment sequences, with a washout phase between doses of at least seven days.

Variables for Evaluation. Primary variables to be examined were Cₘₐₓ, tₘₐₓ, AUC₀₋ₒₒ, AUC₀₋ₜₗₐₛₜ, λ_{z}, t_{1/2}, as derived from non-compartmental pharmacokinetic analysis from the plasma concentration-time curves. Pharmacokinetic parameters of unchanged tolperisone: AUC₍₀₋ₜₗₐₛₜ₎, Cₘₐₓ, and the ratio fed/fasted were evaluated in order to determine the equivalence of food versus non-food treatments.

Safety Considerations. Safety variables and tolerability were assessed by adverse events, safety laboratory (hematology, coagulation, clinical chemistry, and urinalysis), vital signs (BP, HR) and 12-lead ECG.

Statistical Methods: Descriptive Statistics, ANOVA testing for dose proportionality and food interaction were performed.

PK Analysis: Pharmacokinetic characteristics were summarized by the number of measurements, arithmetic mean, standard deviation, coefficient of variation, minimum, median, maximum value and, in addition (tₘₐₓ excluded) by geometric mean, geometric standard deviation (re-transformed standard deviation of logarithms), geometric coefficient of variation and ratio of means and confidence intervals. The logarithms of AUC₀₋ₒₒ and Cₘₐₓ were analyzed by analysis of variance (ANOVA) including sequence, subject (sequence), period, and treatment effects.

Demographics: Descriptive statistics were calculated for demographic parameters such as age, height, weight, and body mass index.

Pre-Study Screening Visit: Prior to the start of the eligibility assessment examinations, the written informed consent form was personally signed and dated by the subject and by the physician who conducted the informed consent discussion. The eligibility assessment examination consisted of the following: medical history, complete physical examination, 12-lead ECG, determination of vital signs (blood pressure, heart rate and body temperature) prior to blood sampling, clinical chemistry, hematology, coagulation, HIV-½Ab, HBsAg, HCV-Ab, urinalysis, urine drug screening, alcohol breath test, and serology.

Hematology and Coagulation: The following parameters were analyzed: hemoglobin, hematocrit, red blood cell count (RBC), white blood cell count (WBC), white differential count, platelet count, prothrombin time (PT), activated partial thromboplastin time (aPTT), mean corpuscular hemoglobin (MCH), mean corpuscular hemoglobin concentration (MCHC), mean corpuscular volume (MCV).

Clinical Chemistry: The following parameters were analyzed: glucose, total cholesterol (differentiated into HDL and LDL), triglycerides, creatinine, uric avid, urea, total protein, alkaline phosphatase (AP), aspartate aminotransferase (AST), alanine aminotransferase (ALT), gamma-glutamyl transpeptidase (γ-GT), creatine phosphokinase (CPK), sodium, potassium, chloride, albumin, calcium.

Urinalysis: Urinalysis (protein, glucose, bilirubin, pH, nitrate, ketone, urobilinogen, blood and leukocytes) was performed using urine dipsticks. The sediment was examined microscopically for erythrocytes, leukocytes, epithelial cells, bacteria, cylinders and crystals.

Serology: Serology analysis included Hepatitis B surface-antigen, hepatitis C virus antibody, HIV-1/Hiv-2 antibodies.

Genotyping: Poor and ultra-fast metabolizers (as a result of CYP 2D6 polymorphism and accounting together for approximately 10% of the population) were excluded.

Urine Drug Screen: Amphetamines, barbiturates, benzodiazepines, cannabinoids, cocaine, and opiate metabolites were screened in urine.

When all eligibility assessment testing procedures had demonstrated that all inclusion criteria and none of the exclusion criteria applied, the subjects were included in the trial. Upon initiation of the trial, subjects were questioned regarding any changes in their health or protocol violations since eligibility assessment.

Treatment Phase: Each treatment period started on the evening of Day-1, approximately 12 h before drug administration and ended approximately 72 h after drug administration (from the evening of Day-1 until the morning of Day-4). During this time the subjects were accommodated at the study center.

On Day-1 the following was performed: urine drug screening, alcohol breath test, and adverse event reporting. The subjects were allowed to eat and drink as usual until approximately 22:00 h on the day prior to the study drug administration (Day-1). Thereafter, the subjects remained fasting (fasting period of 10 hours before administration) with the exception of drinking water, which was only restricted between one (1) hour before and until 2 hours after dosing.

Procedures from Day 1 until Day 3 were as follows (time points are detailed in the tabular schedule of trial procedures):
(i) An indwelling intravenous catheter was placed for blood sampling (without using heparin).
(ii) The study drug was administered (either 150, 300, or 450 mg of tolperisone) with 200 ml non-carbonated water in a standing position in a fasting state (intake of a standard breakfast not earlier than 90 minutes after dosing). For the food effect investigation, subjects received 150 mg tolperisone after food intake.
(iii) 12-lead ECG was taken both pre-dose, and at about 2 h post-dose (+15 min); ECG recordings were performed approximately 15 minutes before blood sampling. The last ECG was performed before discharge on the last day of the last treatment period.
(iv) Blood pressure and heart rate were taken at the following timepoints: Pre-dose, 2, 4, 8, 24, 36, 48, 72 h post-dose, (post-dose±15 min).
(v) Safety laboratory tests were performed at the following timepoints: pre-dose (only Period 1); before discharge on the last day of the last treatment period.
(vi) Blood sampling for pharmacokinetics was carried out as follows. 64 blood samples for the determination of tolperisone and its main metabolite were drawn at the following times: pre-dose and 0.25, 0.5, 0.75, 1.0, 1.25, 1.5, 2, 3, 4, 8, 12, 24, 36, 48, and 72 hours post-administration (16 samples at each treatment).

Actual sampling times were recorded to the minute. The maximum deviations from scheduled sampling times considered irrelevant for pharmacokinetics were defined as shown in Table 1.

TABLE 1
Blood Sampling for Pharmacokinetics

| | Deviation from Scheduled Sampling |
|---|---|
| Blood Sampling Times | (Time considered irrelevant) |
| Pre-dose, Day 1 | −120 min. |
| 0.25 h to 1.5 h | +1 min |
| 2 h to 8 h | +3 min |
| 12 h | +6 min |
| 24 h to 72 h | +15 min |

Unless indicated otherwise, any sample taken earlier than scheduled was considered a time deviation, even if the deviation was irrelevant for the pharmacokinetic evaluation.

Urine Sampling: Urine Sampling for pharmacokinetics was conducted as follows: Pre-dose, 0-4, 4-8, 8-12, 12-24, 24-36, 36-48 h post-dose; subjects collected all excreted urine from the morning of Day 1 pre-dose (last sample voided prior to drug administration) until the morning of Day 3 (48 h post-dose). All voidings within a specific interval were collected into the same container. Date, time interval, weight and density of all urine samples were recorded. A separate container was used if necessary based on the total urine volume. Shortly preceding the end time of a collection interval, subjects were asked to empty their bladder.

Adverse Events: Adverse Event reporting was collected pre-dose to about 48 h post-dose. Approximately 48 h after dosing the subject left the study center, if medically appropriate. Wash-out intervals between dosages lasted for at least 7 days.

General Restrictions and Meals: From 24 h before screening examination and 24 h before intake of the drug of each study period, the consumption of alcohol as well as strenuous exercise were not allowed.

Three days before the screening examination, the consumption of food containing poppy-seed was not allowed. From 24 h before and until 48 h after the administration of the study treatment in each period the consumption of caffeine-containing food and from 48 h before to 48 h after study drug administration grapefruit-containing food or beverages were not allowed. On the day before each study drug administration (Day-1) the subjects were allowed to eat and drink as usual until 22:00 h. After this, they stayed fasting (fasting period of 10 hours before administration) until 4 hours post-dose with the exception of water, which was only restricted before study drug intake until two hours after dosing.

On Day 1, meals were identical for all four study periods, with the exception of the “fed” study period during which a standardized breakfast was served 30 min prior to dosing. During the fasted periods the subjects were served the following meals:

| | |
|---|---|
| Standardized lunch | Four hours p.a. |
| Standardized snack | Seven hours p.a. |
| Standardized dinner | Approx. ten hours p.a. |

In the non-fasted study period subjects received a standardized breakfast 30 min prior to dosing. The breakfast was a high fat breakfast according to FDA recommendations and was composed of the following food items: 2 slices of toasted white bread with butter, 2 eggs fried in butter, 2 slices of bacon, 2 ounces (1 oz=28.35 g) of “hash-browns” (fried, shredded potatoes), and 8 ounces of whole milk.

The nutritional composition of the FDA-recommended high-fat breakfast is summarized in the following table:

TABLE 2
Nutritional Composition of High-Fat Breakfast

| | | | | |
|---|---|---|---|---|
| Carbohydrate | 58 g | 232 kcal | 971 kj | 24% |
| Protein | 33 g | 132 kcal | 552 kj | 14% |
| Fat | 67 g | 603 kcal | 2523 kj | 62% |

Individual Portions were weighed/measured as appropriate. Subjects started consuming the breakfast approximately 30 minutes prior to the scheduled time of dosing and were required to consume all of the breakfast within approximately 20 minutes. The time that the subject started consuming the breakfast, the time that he finished the breakfast, and what, if anything was not eaten was recorded.

Because the subjects were required to consume all of the breakfast, each prospective subject was being shown the menu during the screening interview and asked to confirm that they were able to eat the listed food items. Vegans, vegetarians and other individuals who indicated that they do not normally eat animal products were discouraged from participating in this study. Subjects eating breakfast were supervised, to prevent the exchange of uneaten food items between subjects. In addition, those subjects receiving breakfast were served their food away from those subjects who were required to remain fasted.

Beverages were standardized from Day 1 until the morning of Day 3. The subjects were required to drink 105 to 2.5 liter per day equally distributed over the day. The beverage restriction was controlled and documented by the study personnel. Until 12 h after the study drug administration the subjects were allowed to drink 200 ml every 2 hours.

End of Study Examinations: The end-of-study examinations verified that all values tested during the eligibility assessment remained within a clinically acceptable range. These examinations took place at the end of the last day of the last treatment period and comprised the following: physical examination, 12-lead ECG, vital signs (blood pressure, heart rate), safety laboratory and urinalysis in the morning fasted state, and adverse event reporting.

Blood Sampling Schedule: In total, approximately 450 milliliters of blood were collected for each subject during the study. All samples were drawn according to the study schedule and processed according to the respective standard procedures. The label on each tube stated study number, subject number, sampling time, and date.

Assessment of Pharmacokinetics: For pharmacokinetic analysis, 7 ml of blood were collected by using Sarstedt monovettes (Sarstedt Ltd., Leicester, UK) or Vacutainers (Becton Dickenson).

The samples were immediately stored in ice water and centrifuged within 30 min following the collection of blood (1500×g, 10 minutes) at +4° C. temperature. The plasma from each sample was divided into two equal aliquots (approx. 2×1.5 ml), which were each transferred to a polypropylene tube, and identified by a coded label. The plasma samples were then stored in a freezer at a temperature of at least −70° C. until time of analysis.

The frozen samples were shipped to the analytical laboratory in appropriate containers with a sufficient amount of dry ice and analyzed for drug concentration. Analysis was carried out using a validated HPLC-method.

Pharmacokinetic Analyses: For the non-compartmental pharmacokinetic analysis WinNonlin® (Windows Non-Linear PK software version 3.1) was used as software. Real times of blood sampling were used for the analysis.

From each plasma concentration-time curve of tolperisone and its main metabolite the following pharmacokinetic variables were determined provided that concentrations were quantifiable: Cₘₐₓ (observed maximum concentration), tₘₐₓ (time of observed maximum concentration), AUC₀₋ₜₗₐₛₜ (area under the plasma concentration-time curve extrapolated from the time of the last quantifiable plasma concentration to infinity=Cₜₗₐₛₜ/λ_{z}), AUC₀₋ₒₒ (area under the plasma concentration-time curve from time zero to infinity; =AUC₀₋ₜₗₐₛₜ+AUCₜₗₐₛₜ₋ₒₒ); λ (apparent terminal rate constant derived from the slope of the log-linear regression of the log-linear terminal portion of the plasma-concentration time curve); t_{1/2} (apparent terminal plasma half-life;=In2/λ_{z}). AUC was determined by linear trapezoidal rule, AUC₍₀₋ₒₒ₎=AUC_{(0-tlastZ})+C_{z}/λ_{z} where C_{z} was the last quantifiable concentration (the extrapolation was considered unreliable if the terminal area beyond the last quantified sample was greater than 20% of the total AUC₍₀₋ₒₒ₎, λ_{z} was determined by log-linear regression, t_{1/2}=(In 2)/λ_{z}, CL_{zt}/F=dose/AUC₍₀₋ₒₒ₎; F=ratio AUC₍₀₋ₒₒ₎ following non-food and food dose expressed in %.

From concentrations in urine and urine volumes the amounts of tolperisone and its main metabolite excreted were determined and the following pharmacokinetic parameters calculated: Aeᵤᵣ (total amount of drug excreted into urine (ng/ml)); Clrₑₙ (renal clearance).

TABLE 3
Randomization Scheme

| Treatment | Rand. | | | | |
|---|---|---|---|---|---|
| Group | No. | Period 1 | Period 2 | Period 3 | Period 4 |
| A | 3 | 150 mg | 450 mg | 300 mg | 150 mg - with food |
| | 6 | 150 mg | 450 mg | 300 mg | 150 mg - with food |
| | 10 | 150 mg | 450 mg | 300 mg | 150 mg - with food |
| | 15 | 150 mg | 450 mg | 300 mg | 150 mg - with food |
| | 16 | 150 mg | 450 mg | 300 mg | 150 mg - with food |
| | 18 | 150 mg | 450 mg | 300 mg | 150 mg - with food |
| B | 7 | 450 mg | 150 mg - with food | 150 mg | 300 mg |
| | 9 | 450 mg | 150 mg - with food | 150 mg | 300 mg |
| | 11 | 450 mg | 150 mg - with food | 150 mg | 300 mg |
| | 20 | 450 mg | 150 mg - with food | 150 mg | 300 mg |
| | 22 | 450 mg | 150 mg - with food | 150 mg | 300 mg |
| | 24 | 450 mg | 150 mg - with food | 150 mg | 300 mg |
| | 920 | 450 mg | 150 mg - with food | 150 mg | 300 mg |
| C | 2 | 300 mg | 150 mg | 150 mg - with food | 450 mg |
| | 5 | 300 mg | 150 mg | 150 mg - with food | 450 mg |
| | 8 | 300 mg | 150 mg | 150 mg - with food | 450 mg |
| | 13 | 300 mg | 150 mg | 150 mg - with food | 450 mg |
| | 14 | 300 mg | 150 mg | 150 mg - with food | 450 mg |
| | 19 | 300 mg | 150 mg | 150 mg - with food | 450 mg |
| D | 1 | 150 mg - with food | 300 mg | 450 mg | 150 mg |
| | 4 | 150 mg - with food | 300 mg | 450 mg | 150 mg |
| | 12 | 150 mg - with food | 300 mg | 450 mg | 150 mg |
| | 17 | 150 mg - with food | 300 mg | 450 mg | 150 mg |
| | 21 | 150 mg - with food | 300 mg | 450 mg | 150 mg |
| | 23 | 150 mg - with food | 300 mg | 450 mg | 150 mg |
| | 921 | 150 mg - with food | 300 mg | 450 mg | 150 mg |

Pharmacokinetic Results: The concentrations of total unchanged tolperisone, and its main metabolite, 4-hydroxymethyl-tolperisone (4-HM-tolperisone), in blood plasma samples were determined through GC/MC and LC/MC/MS methods. The analysis of the pharmacokinetic data was preformed using non-compartmental methods in WinNonlin® version 3.1.

The PK parameters assessed by descriptive statistics included observed peak plasma concentration (Cₘₐₓ), time of observed peak plasma concentration (tₘₐₓ), area under the plasma concentration time curve from time zero to the last quantifiable time point (AUC₀₋ₜₗₐₛₜ), area under the plasma concentration time curve from time zero to infinity (AUC0-oo), the terminal phase elimination rate constant (λ_{z}) and terminal elimination half-life (t_{1/2}). The tables below summarize the main pharmacokinetic characteristics for both unchanged and metabolized tolperisone.

Plasma concentrations of total unchanged tolperisone and its main metabolite, 4-HM-tolperisone, were determined for all treatment groups in all study periods. Blood sampling times were 2 h pre-dose and 0.25, 0.5, 0.75, 1, 1.25, 1.5, 2, 3, 4, 8, 12, 24, 36, 48, and 72 h post-dose. The concentrations of 4-HM-tolperisone in blood plasma in all studied doses (150, 300, and 450 mg) were below the limit of quantification eight hours after study drug administration. These values were ignored and were not used for the calculations of pharmacokinetic parameters.

Mean plasma concentrations of tolperisone and 4-HM-tolperisone are shown in FIG. 1 and FIG. 2, respectively, for the different treatment groups.

The effect of food on the pharmacokinetics of tolperisone and 4-HM-tolperisone was assessed by comparison of fed vs. fasted state. As described in detail above, the subjects received a single dose of 150 mg tolperisone coated tablets either in a fasted state or after a high fat standard meal. This part of the study was performed because a food effect could not be excluded due to the high lipophilic properties of the substance.

The results of the plasma pharmacokinetic variables are summarized in the following two tables.

TABLE 4
Pharmacokinetic Parameters of Unchanged Total Tolperisone (Mean ± SD) in Fasted
and Fed States

| | Parameters | | | | | |
|---|---|---|---|---|---|---|
| Dose | Cₘₐₓ | tₘₐₓ | AUC₀₋ₜₗₐₛₜ | AUC₀₋ₒₒ | λ_{z} | t_{1/2} |
| (mg) | (ng/mL) | (h) | Ng h/mL) | (ng h/mL) | (1/h) | (h) |
| 150 | 36.1 ± 38.4 | 0.66 ± 0.16 | 54.0 ± 53.2 | 55.4 ± 53.4 | 0.35 ± 0.08 | 2.06 ± 0.43 |
| fasted | | | | | | |
| state | | | | | | |
| 150 fed | 40.2 ± 37.4 | 1.38 ± 0.71 | 87.4 ± 74.7 | 89.3 ± 75.1 | 0.30 ± 0.11 | 2.84 ± 1.83 |
| state | | | | | | |

TABLE 5
Pharmacokinetic Parameters of Total 4-HM-Tolperisone (Mean ± SD) in Fasted and
Fed States

| | Parameters | | | | | |
|---|---|---|---|---|---|---|
| Dose | Cₘₐₓ | tₘₐₓ | AUC₀₋ₜₗₐₛₜ | AUC₀₋ₒₒ | λ_{z} | t_{1/2} |
| (mg) | (ng/mL) | (h) | Ng h/mL) | (ng h/mL) | (1/h) | (h) |
| 150 | 1.70 ± 1.66 | 0.68 ± 0.23 | 1.20 ± 1.01 | 1.82 ± 1.09 | 1.45 ± 0.51 | 0.55 ± 0.21 |
| fasted | | | | | | |
| state | | | | | | |
| 150 fed | 2.56 ± 3.04 | 1.40 ± 0.77 | 2.69 ± 2.18 | 3.92 ± 2.37 | 0.95 ± 0.90 | 1.19 ± 0.80 |
| state | | | | | | |

Point estimates and corresponding 90% confidence intervals (Cls) for the comparison of fed/fasted conditions are presented in the following table.

TABLE 6
Geometric Mean for the Ratio 150 mg Fed/150 mg Fasted

| | | | |
|---|---|---|---|
| Parameter | Comparison | Point Estimate | 90% Cl |

Tolperisone

| | | | |
|---|---|---|---|
| AUC₀₋ₒₒ | Fed/fasted | 1.87 | (1.48, 2.35) |
| AUC₀₋ₜₗₐₛₜ | Fed/fasted | 1.92 | (1.50, 2.44) |
| Cₘₐₓ | Fed/fasted | 1.18 | (0.84, 1.66) |

4-HM-Tolperisone

| | | | |
|---|---|---|---|
| AUC₀₋ₒₒ | Fed/fasted | 1.95 | (1.58, 2.40) |
| AUC₀₋ₜₗₐₛₜ | Fed/fasted | 2.27 | (1.61, 3.22) |
| Cₘₐₓ | Fed/fasted | 1.23 | 0.83, 1.81) |

The calculated data clearly indicated a food effect. AUC and Cₘₐₓ (geometric mean for the ratio 150 mg fed/150 mg fasted) in the fed state were approximately two-fold higher compared to the fasted state.

In the fasted state, tₘₐₓ values of unchanged tolperisone and 4-HM-tolperisone were 0.66 h (+0.16 h) and 0.68 h (+0.23 h), respectively. In the fed state, tₘₐₓ values of unchanged tolperisone and 4-HM-tolperisone were 1.38 h (±0.77 h), indicating a delayed absorption after food intake.

The bioavailability was higher under fed conditions compared to fasted conditions. The point estimate for AUC₀₋ₒₒ, and AUC₀₋ₜₗₐₛₜ (fed/fasted) were 1.87 and 1.92, respectively, for tolperisone. The effect of food was less pronounced for Cₘₐₓ, with a point estimate of 1.18 for tolperisone. Absorption was delayed with a mean tₘₐₓ of 1.37 hours in the fed state compared to 0.66 hours in the fasted state.

### Example 2

### Pharmacokinetic profile of Tolperisone After Single and Multi-Dosing

An objective of this study was to explore the pharmacokinetic profile of tolperisone after single and multi-dosing.

Subjects: Each cohort, fasted and fed, consisted of 15 subjects, 10 randomized to receive active drug and five (5) randomized to receive placebo. A total of 30 subjects were enrolled, 20 randomized to active drug and 10 to placebo. All subjects randomized to active drug completed the study. One placebo subject withdrew from the study.

Dosing Regimen: Subjects in each cohort received tolperisone according to the following regimen.

TABLE 7
Dosing Regimen for Tolperisone
Dose¹ and Dosing Time

| Day | 0800 | 1400 | 2000 |
|---|---|---|---|
| 1 | 150 mg | Placebo | Placebo |
| 2 | 150 mg | Placebo | 150 mg |
| 3 | 150 mg | 150 mg | 150 mg |
| 4 | 150 mg | 150 mg | 150 mg |
| 5 | 150 mg | 150 mg | 150 mg |
| 6 | 150 mg | 150 mg | 150 mg |
| 7 | 150 mg | 150 mg | 150 mg |

¹Subjects randomized to placebo received placebo at all three dosing times.

Blood samples for the measurement of the plasma concentrations of tolperisone and metabolites M1 (4-hydroxymethyltolperisone) and M4 (4-carboxy-tolperisone) were collected at the times shown in Table 8.

TABLE 8
Blood Sampling Schedule

| Day | Dose | Sample Times(h)¹ |
|---|---|---|
| 1 | 0800 | 0, 0.25, 0.5, 0.75, 1, 1.5, 2, 3, and 6 |
| 3 | 0800 | 0 |
| | 2000 | 0, 0.5, 0.75, 1, 1.5 |
| 5 | 0800 | 0 |
| 7 | 0800 | 0, 0.25, 0.5, 0.75, 1, 1.5, 2, 3, and 6² |
| | 2000 | 0, 0.5, 0.75, 1, 1.5, 12 |

¹”0” is immediately prior to dosing.
²Collected immediately prior to the dose at 1400.

Bioanalytical: Plasma concentrations of tolperisone and metabolites M1 and M4 were measured using a validated LC/MS/MS assay by Pharm-analyt Laboratory, Baden Austria. The limits of quantitation (LOQ) for tolperisone, Mi, and M4 in plasma were 0.2 ng/ML, and 0.01 μg/mL, respectively.

Pharmacokinetic Analyses: Pharmacokinetic parameters for tolperisone, M1, and M4 were calculated using non-compartmental analysis. Only plasma concentrations greater than or equal to the LOQ for the respective assay were used in the pharmacokinetic analyses. Actual blood sampling times were used in all pharmacokinetic analyses. Per protocol times were used to calculate mean plasma concentrations for graphical displays. All pharmacokinetic calculations were done using SAS® for Windows® Version 9.1.3.

Day 1—0800 Dose

The maximum plasma concentration (Cₘₐₓ) and time to Cₘₐₓ (Tₘₐₓ) were taken directly from the data. The elimination rate constant, λz, was calculated as the negative of the slope of the terminal log-linear segment of the plasma concentration-time curve. The range of data to be used for each subject and treatment was determined by visual inspection of a semi-logarithmic plot of concentration versus time. Elimination half-life (t½) was calculated according to the following equation:

$$t3 / 2 = \frac{0.693}{λ z}$$

Area under the curve to the final sample with a concentration greater than or equal to the LOQ [(AUC(0-t)] was calculated using the linear trapezoidal method and extrapolated to infinity [AUC(inf)] using:

$$AUC ( inf ) = AUC ( 0 - t ) + \frac{{C}_{tf}}{λ z}$$

where C_{tf} is the final concentration≧LOQ.

Day 3—2000 Dose

The maximum plasma concentration (Cₘₐₓ) and time to Cₘₐₓ (Tₘₐₓ) were taken directly from the data.

Day 7—0800 Dose

The maximum plasma concentration (Cₘₐₓ) and time to Cₘₐₓ (Tₘₐₓ) were taken directly from the data. The elimination rate constant, λz, and half-life, t½, were calculated as described for the 0800 dose on Day 1 (above). Areas under the curve to the final sample with a concentration greater than or equal to the LOQ [(AUC(O-t)] and over the 6-hour dosing interval [AUC(0-6)] were calculated using the linear trapezoidal method.

Day 7—2000 Dose

The maximum plasma concentration (Cₘₐₓ) and time to Cₘₐₓ (Tₘₐₓ) were taken directly from the data.

### Results

A. Both Sexes Combined

Tolperisone: Administration of tolperisone with food resulted in an apparent increase in absorption on Day 1 as evidenced by increases of 23% and 39% in the mean values for Cₘₐₓ and AUC(inf) (Table 9); however, by Day 7, absorption after the 0800 dose under fasted conditions was greater with a mean Cₘₐₓ that was 213% higher and a mean AUC(0-6) that was 127% higher (Table 9). This decrease under fed conditions was also evident from the mean plasma concentrations and values for Cₘₐₓ after the 2000 dose on Day 7 (FIG. 3; Table 9). Whether this apparent discordance in the results represents a real phenomenon may be clarified by examining the effect of food using a crossover design.

Mean values for t½ ranged from 1.22 to 1.59 hours and did not appear to be dependent upon administration with food or the duration of dosing (Table 9). Consistent with the t½ and the dosing frequency, and taking into consideration the number of subjects and the variability, there did not appear to be an increase in Cₘₐₓ over the seven days of dosing, suggesting little or no accumulation.

Metabolite M1: Consistent with the parent, administration of tolperisone with food resulted in an apparent increase in exposure to Metabolite M1 on Day 1 as evidenced by increases of 42% and 61% in the mean values for Cₘₐₓ and AUC(inf) (Table 10); however, by Day 7, exposure after the 0800 dose under fasted and fed conditions was more comparable with a mean Cₘₐₓ under fed conditions that was 12% lower and a mean AUC(0-6) that was 12% higher (Table 10). The comparable exposure under fasted and fed conditions was also evident from the mean plasma concentrations and values for Cₘₐₓ after the 2000 dose on Day 7 (Table 10).

Mean values for t½ ranged from 1.03 to 1.18 hours and did not appear to be dependent upon administration with food or the duration of dosing (Table 10). Consistent with the t½ and the dosing frequency, and taking into consideration the number of subjects and the variability, there did not appear to be an increase in Cₘₐₓ over the seven days of dosing, suggesting little or no accumulation.

Metabolite M4: Administration of tolperisone with food resulted in minimal changes in exposure to Metabolite M4 on Day 1 as evidenced by a decrease of 11% and an increase of 6% in the mean values for Cₘₐₓ and AUC(inf) (Table 11). Similarly, on Day 7, exposure after the 0800 dose under fasted and fed conditions was comparable with a mean Cₘₐₓ under fed conditions that was 28% lower and a mean AUC(0-6) that was 3% lower (Table 11). The comparable exposure under fasted and fed conditions was also evident from the mean plasma concentrations and values for Cₘₐₓ after the 2000 dose on Day 7 (Table 11).

Mean values for t½ ranged from 1.07 to 1.17 hours and did not appear to be dependent upon administration with food or the duration of dosing (Table 11). Consistent with the t½ and the dosing frequency, and taking into consideration the number of subjects and the variability, there did not appear to be an increase in Cₘₐₓ over the seven days of dosing, suggesting little or no accumulation.

B. By Sex

There were seven males and three females in the fasted cohort and six males and four females in the fed cohort.

Tolperisone: Mean plasma concentrations appeared to be higher in females than in males for the entire seven-day treatment period and for Days 1 and 7 after administration under both fasted and fed conditions. The higher exposure in females was also evident from the mean values for Cₘₐₓ and AUC (Table 12). The apparent increase in the absorption of tolperisone when administered with food on Day 1 and decrease on Day 7 observed with the combined data may be due to the female in the fed cohort rather than the males (Table 12) and suggests that the overall effect of food is to decrease the absorption of tolperisone.

Mean values for t½ ranged from 1.18 to 1.62 hours in males and from 1.17 to 1.53 hours in females and did not appear to be dependent upon sex, administration with food or the duration of dosing (Table 12).

Metabolite M1: With the exception of females in the fed cohort on Day 1 and the fasted cohort on Day 7, mean plasma concentrations were comparable in males and females. This was also evident from the mean values for Cₘₐₓ and AUC (Table 13). However, these differences may be more due to the small numbers of subjects than to a difference between males and females. Overall, it appears that exposure to Metabolite M1 is not different between males and females and not affected by food.

Mean values for t½ ranged from 1.03 to 1.23 hours in males and from 1.03 to 1.08 hours in females and did not appear to be dependent upon sex, administration with food or the duration of dosing (Table 13).

Metabolite M4: Mean plasma concentrations were comparable in males and females as were the mean values for Cₘₐₓ and AUC (Table 14). Although mean plasma concentrations of M4 suggested an effect of food on Day 7, examination of the mean values for Cₘₐₓ and AUC (Table 14) indicated that the effect was minimal. Overall, it appeared that exposure to Metabolite M4 is not different between males and females and not affected by food.

Mean values for t½ ranged from 1.03 to 1.17 hours in males and from 0.99 to 1.16 hours in females and did not appear to be dependent upon sex, administration with food or the duration of dosing (Table 14).

To summarize the results of this experiment, administration of tolperisone under fed conditions appeared to decrease the extent of absorption of the parent but had minimal effect on the exposure to Metabolites M1 and M4. The mean plasma concentrations and Cₘₐₓ and AUC of tolperisone appeared to be higher in female subjects than in male subjects under fasted and fed conditions. However, exposure to Metabolites M1 and M4 appeared to be comparable in both sexes. The mean elimination half-lives of tolperisone, M1, and M4 were approximately 1.3, 1.1, and 1.1 hours, respectively, and were not dependent on sex, administration of food, or the duration of dosing. Consistent with the t½ and dosing frequency, there did not appear to be an increase in the mean Cₘₐₓ of tolperisone, M1, and M4 over the seven days of dosing, suggesting little or no accumulation.

Thus, methods for administering tolperisone are described. Although preferred embodiments of the subject invention have been described in some detail, it is understood that obvious variations can be made without departing from the spirit and the scope of the invention as claimed herein.

TABLE 9
Summary of Pharmacokinetic Parameters for Tolperisone
After Oral Administration of Tolperisone on a Regimen of
150 mg at 0800 on Day 1, 150 mg at 0800 and 2000 on
Day 2, and 150 mg at 0800, 1400 and 2000 on Days 3
through 7 to Healthy Subjects Under Fasted
and Fed Conditions

| Parameter¹ | Fasted | Fed |
|---|---|---|
| Day 1 - 0800 Dose | | |
| Cmax (ng/mL) | 75.4 ± 62.2 (10) | 92.5 ± 109 (10) |
| Tmax (h) | 0.88 (10) | 1.00 (10) |
| AUC (0-t) (h · ng/mL) | 117 ± 93.0 (10) | 161 ± 179 (10) |
| AUC (inf) (h · ng/mL) | 122 ± 97.5 (10) | 169 ± 187 (10) |
| λz (h⁻¹) | 0.5887 ± 0.1095 (10) | 0.5676 ± 0.0914 (10) |
| t½ (h) | 1.22 ± 0.24 (10) | 1.25 ± 0.19 (10) |
| Day 2 - 2200 Dose | | |
| Cmax (ng/mL) | 40.9 ± 33.9 (10) | 42.3 ± 55.1 (10) |
| Tmax (h) | 1.25 (10) | 1.50 (10) |
| Day 7 - 0800 Dose | | |
| Cmax (ng/mL) | 133 ± 110 (10) | 62.5 ± 50.3 (10) |
| Tmax (h) | 0.75 (10) | 1.25 (10) |
| AUC (0-6) (h · ng/mL) | 196 ± 128.9 (10) | 156 ± 159 (10) |
| λz (h⁻¹) | 0.4722 ± 0.1096 (10) | 0.5726 ± 0.1095 (10) |
| t½ (h) | 1.59 ± 0.63 (10) | 1.26 ± 0.28 (10) |
| Day 7 - 2200 Dose | | |
| Cmax (ng/mL) | 76.4 ± 63.5 (10) | 55.5 ± 63.7 (10) |
| Tmax (h) | 1.00 (10) | 1.00 (10) |

¹Mean ± standard deviation (N) except for Tmax for which the median (N) is reported.

TABLE 10
Summary of Pharmacokinetic Parameters for Metabolite M1
After Oral Administration of Tolperisone on a Regimen of
150 mg at 0800 on Day 1, 150 mg at 0800 and 2000 on
Day 2, and 150 mg at 0800, 1400 and 2000 on Days 3 through
7 to Healthy Subjects Under Fasted and Fed Conditions

| Parameter¹ | Fasted | Fed |
|---|---|---|
| Day 1 - 0800 Dose | | |
| Cmax (ng/mL) | 2.22 ± 1.40 (10) | 3.16 ± 1.46 (10) |
| Tmax (h) | 0.88 (10) | 1.00 (10) |
| AUC (0-t) (h · ng/mL) | 3.63 ± 1.69 (10) | 5.82 ± 3.86 (10) |
| AUC (inf) (h · ng/mL) | 3.75 ± 1.72 (10) | 6.05 ± 4.11 (10) |
| λz (h⁻¹) | 0.6293 ± 0.0788 (10) | 0.6754 ± 0.0599 (10) |
| t½ (h) | 1.12 ± 0.17 (10) | 1.03 ± 0.10 (10) |
| Day 2 - 2200 Dose | | |
| Cmax (ng/mL) | 1.30 ± 0.74 (10) | 1.64 ± 1.33 (10) |
| Tmax (h) | 1.25 (10) | 1.00 (10) |
| Day 7 - 0800 Dose | | |
| Cmax (ng/mL) | 2.83 ± 1.63 (10) | 2.49 ± 1.61 (10) |
| Tmax (h) | 0.75 (10) | 1.00 (10) |
| AUC (0-6) (h · ng/mL) | 4.63 ± 2.33 (10) | 5.20 ± 3.25 (10) |
| λz (h⁻¹) | 0.6054 ± 0.0982 (10) | 0.6789 ± 0.1437 (08) |
| t½ (h) | 1.18 ± 0.21 (10) | 1.08 ± 0.31 (08) |
| Day 7 - 2200 Dose | | |
| Cmax (ng/mL) | 2.27 ± 1.64 (10) | 2.20 ± 1.51 (10) |
| Tmax (h) | 1.00 (10) | 1.00 (10) |

¹Mean ± standard deviation (N) except for Tmax for which the median (N) is reported.

TABLE 11
Summary of Pharmacokinetic Parameters for Metabolite M4
After Oral Administration of Tolperisone on a Regimen of
150 mg at 0800 on Day 1, 150 mg at 0800 and 2000 on
Day 2, and 150 mg at 0800, 1400 and 2000 on Days 3 through
7 to Healthy Subjects Under Fasted and Fed Conditions

| Parameter¹ | Fasted | Fed |
|---|---|---|
| Day 1 - 0800 Dose | | |
| Cmax (μg/mL) | 1.31 ± 0.36 (10) | 1.17 ± 0.38 (10) |
| Tmax (h) | 0.88 (10) | 1.50 (10) |
| AUC (0-t) (h · μg/mL) | 2.42 ± 0.46 (10) | 2.49 ± 0.70 (10) |
| AUC (inf) (h · μg/mL) | 2.49 ± 0.48 (10) | 2.64 ± 0.79 (10) |
| λz (h⁻¹) | 0.6519 ± 0.0524 (10) | 0.6104 ± 0.1067 (10) |
| t½ (h) | 1.07 ± 0.088 (10) | 1.16 ± 0.19 (10) |
| Day 2 - 2200 Dose | | |
| Cmax (μg/mL) | 0.66 ± 0.40 (10) | 0.78 ± 0.40 (10) |
| Tmax (h) | 1.25 (10) | 1.00 (10) |
| Day 7 - 0800 Dose | | |
| Cmax (μg/mL) | 1.19 ± 0.38 (10) | 0.86 ± 0.32 (10) |
| Tmax (h) | 0.75 (10) | 2.00 (10) |
| AUC (0-6) (h · μg/mL) | 2.30 ± 0.54 (10) | 2.23 ± 0.49 (10) |
| λz (h⁻¹) | 0.5934 ± 0.0406 (10) | 0.6136 ± 0.1092 (09) |
| t½ (h) | 1.17 ± 0.081 (10) | 1.17 ± 0.26 (09) |
| Day 7 - 2200 Dose | | |
| Cmax (μg/mL) | 1.01 ± 0.35 (10) | 0.96 ± 0.24 (10) |
| Tmax (h) | 1.27 (10) | 1.04 (10) |

¹Mean ± standard deviation (N) except for Tmax for which the median (N) is reported.

TABLE 12
Summary of Pharmacokinetic Parameters for Tolperisone by Sex After Oral
Administration of Tolperisone on a Regimen of 150 mg at 0800 on Day 1, 150 mg
at 0800 and 2000 on Day 2, and 150 mg at 0800, 1400 and 2000 on Days 3 through 7 to
Healthy Subjects Under Fasted and Fed Conditions

| | Fasted | | Fed | |
|---|---|---|---|---|
| Parameter¹ | Males | Females | Males | Females |
| Day 1 - 0800 Dose | | | | |
| Cmax (ng/mL) | 66.6 ± 51.6 (7) | 96.0 ± 92.5 (3) | 57.5 ± 20.8 (6) | 145 ± 170 (4) |
| Tmax (h) | 0.75 (7) | 1.00 (3) | 0.88 (6) | 1.50 (4) |
| AUC(0-t) (h · ng/mL) | 93.1 ± 55.2 (7) | 173 ± 152 (3) | 81.7 ± 32.0 (6) | 280 ± 251 (4) |
| AUC(inf) (h · ng/mL) | 96.3 ± 55.7 (7) | 181 ± 161 (3) | 85.7 ± 35.1 (6) | 295 ± 260 (4) |
| λz (h⁻¹) | 0.5785 ± 0.1114 (7) | 0.6127 ± 0.1246 (3) | 0.5510 ± 0.0927 (6) | 0.5924 ± 0.0969 (4) |
| t½ (h) | 1.24 ± 0.25 (7) | 1.17 ± 0.25 (3) | 1.28 ± 0.19 (6) | 1.20 ± 0.21 (4) |
| Day 2 - 2200 Dose | | | | |
| Cmax (ng/mL) | 30.4 ± 25.8 (7) | 65.3 ± 43.4 (3) | 22.4 ± 16.8 (6) | 72.2 ± 81.7 (4) |
| Tmax (h) | 1.50 (7) | 0.75 (3) | 1.50 (6) | 1.25 (4) |
| Day 7 - 0800 Dose | | | | |
| Cmax (ng/mL) | 106 ± 100 (7) | 197 ± 125 (3) | 42.7 ± 23.5 (6) | 92.2 ± 68.7 (4) |
| Tmax (h) | 0.75 (7) | 0.75 (3) | 1.25 (6) | 1.38 (4) |
| AUC(0-6) (h · ng/mL) | 162 ± 113 (7) | 276 ± 151 (3) | 97.4 ± 50.6 (6) | 244 ± 234 (4) |
| λz (h⁻¹) | 0.4807 ± 0.1325 (7) | 0.4525 ± 0.0234 (3) | 0.6069 ± 0.1105 (6) | 0.5211 ± 0.0987 (4) |
| t½ (h) | 1.62 ± 0.77 (7) | 1.53 ± 0.08 (3) | 1.18 ± 0.28 (6) | 1.37 ± 0.26 (4) |
| Day 7 - 2200 Dose | | | | |
| Cmax (ng/mL) | 57.7 ± 46.8 (7) | 120 ± 86.8 (3) | 30.6 ± 18.5 (6) | 92.8 ± 92.2 (4) |
| Tmax (h) | 1.03 (7) | 1.00 (3) | 0.88 (6) | 1.00 (4) |

¹Mean ± standard deviation (N) except for Tmax for which the median (N) is reported.

TABLE 13
Summary of Pharmacokinetic Parameters for Metabolite M1 by Sex After Oral
Administration of Tolperisone on a Regimen of 150 mg at 0800 on Day 1, 150 mg
at 0800 and 2000 on Day 2, and 150 mg at 0800, 1400 and 2000 on Days 3 through 7 to
Healthy Subjects Under Fasted and Fed Conditions

| | Fasted | | Fed | |
|---|---|---|---|---|
| Parameter¹ | Males | Females | Males | Females |
| Day 1 - 0800 Dose | | | | |
| Cmax (ng/mL) | 2.30 ± 1.50 (7) | 2.06 ± 1.44 (3) | 3.07 ± 0.68 (6) | 3.29 ± 2.36 (4) |
| Tmax (h) | 0.75 (7) | 1.00 (3) | 0.88 (6) | 2.00 (4) |
| AUC(0-t) (h · ng/mL) | 3.49 ± 1.47 (7) | 3.98 ± 2.48 (3) | 4.22 ± 1.08 (6) | 8.23 ± 5.46 (4) |
| AUC(inf) (h · ng/mL) | 3.60 ± 1.48 (7) | 4.10 ± 2.55 (3) | 4.31 ± 1.12 (6) | 8.64 ± 5.79 (4) |
| λz (h⁻¹) | 0.6081 ± 0.0792 (7) | 0.6787 ± 0.0627 (3) | 0.6791 ± 0.0554 (6) | 0.6700 ± 0.0749 (4) |
| t½ (h) | 1.16 ± 0.18 (7) | 1.03 ± 0.090 (3) | 1.03 ± 0.09 (6) | 1.04 ± 0.12 (4) |
| Day 2 - 2200 Dose | | | | |
| Cmax (ng/mL) | 1.08 ± 0.68 (7) | 1.83 ± 0.70 (3) | 1.24 ± 0.60 (6) | 2.23 ± 1.99 (4) |
| Tmax (h) | 1.50 (7) | 0.75 (3) | 1.00 (6) | 1.25 (4) |
| Day 7 - 0800 Dose | | | | |
| Cmax (ng/mL) | 2.42 ± 1.62 (7) | 3.79 ± 1.46 (3) | 1.70 ± 0.67 (6) | 3.67 ± 1.97 (4) |
| Tmax (h) | 0.75 (7) | 0.75 (3) | 1.00 (6) | 1.13 (4) |
| AUC(0-6) (h · ng/mL) | 4.13 ± 2.37 (7) | 5.78 ± 2.18 (3) | 3.82 ± 1.23 (6) | 7.28 ± 4.43 (4) |
| λz (h⁻¹) | 0.5766 ± 0.0978 (7) | 0.6728 ± 0.0705 (3) | 0.6791 ± 0.1380 (6) | 0.6783 ± 0.2222 (2) |
| t½ (h) | 1.23 ± 0.22 (7) | 1.04 ± 0.115 (3) | 1.08 ± 0.32 (6) | 1.08 ± 0.35 (2) |
| Day 7 - 2200 Dose | | | | |
| Cmax (ng/mL) | 1.78 ± 1.23 (7) | 3.43 ± 2.15 (3) | 2.09 ± 1.33 (6) | 2.35 ± 1.97 (4) |
| Tmax (h) | 1.03 (7) | 1.00 (3) | 0.88 (6) | 1.25 (4) |

¹Mean ± standard deviation (N) except for Tmax for which the median (N) is reported.

TABLE 14
Summary of Pharmacokinetic Parameters for Metabolite M4 by Sex After Oral
Administration of Tolperisone on a Regimen of 150 mg at 0800 on Day 1, 150 mg
at 0800 and 2000 on Day 2, and 150 mg at 0800, 1400 and 2000 on Days 3 through 7 to
Healthy Subjects Under Fasted and Fed Conditions

| | Fasted | | Fed | |
|---|---|---|---|---|
| Parameter¹ | Males | Females | Males | Females |
| Day 1 - 0800 Dose | | | | |
| Cmax (μg/mL) | 1.31 ± 0.44 (7) | 1.31 ± 0.05 (3) | 1.27 ± 0.45 (6) | 1.02 ± 0.18 (4) |
| Tmax (h) | 0.75 (7) | 1.00 (3) | 0.88 (6) | 2.00 (4) |
| AUC(0-t) (h · μg/mL) | 2.26 ± 0.42 (7) | 2.79 ± 0.37 (3) | 2.32 ± 0.70 (6) | 2.74 ± 0.74 (4) |
| AUC(inf) (h · μg/mL) | 2.34 ± 0.44 (7) | 2.86 ± 0.40 (3) | 2.43 ± 0.76 (6) | 2.94 ± 0.84 (4) |
| λz (h⁻¹) | 0.6321 ± 0.0488 (7) | 0.6981 ± 0.0253 (3) | 0.6047 ± 0.0938 (6) | 0.6189 ± 0.1389 (4) |
| t½ (h) | 1.10 ± 0.08 (7) | 0.99 ± 0.04 (3) | 1.17 ± 0.19 (6) | 1.16 ± 0.21 (4) |
| Day 2 - 2200 Dose | | | | |
| Cmax (μg/mL) | 0.53 ± 0.32 (7) | 0.96 ± 0.48 (3) | 0.75 ± 0.42 (6) | 0.82 ± 0.41 (4) |
| Tmax (h) | 1.50 (7) | 1.00 (3) | 1.00 (6) | 1.25 (4) |
| Day 7 - 0800 Dose | | | | |
| Cmax (μg/mL) | 1.04 ± 0.32 (7) | 1.57 ± 0.25 (3) | 0.80 ± 0.24 (6) | 0.95 ± 0.45 (4) |
| Tmax (h) | 0.75 (7) | 0.75 (3) | 1.75 (6) | 2.00 (4) |
| AUC(0-6) (h · μg/mL) | 2.06 ± 0.39 (7) | 2.86 ± 0.42 (3) | 2.17 ± 0.57 (6) | 2.31 ± 0.39 (4) |
| λz (h⁻¹) | 0.5809 ± 0.0358 (7) | 0.6226 ± 0.0416 (3) | 0.5978 ± 0.1116 (6) | 0.6452 ± 0.1199 (3) |
| t½ (h) | 1.20 ± 0.07 (7) | 1.12 ± 0.08 (3) | 1.21 ± 0.30 (6) | 1.10 ± 0.20 (3) |
| Day 7 - 2200 Dose | | | | |
| Cmax (μg/mL) | 0.844 ± 0.243 (7) | 1.40 ± 0.21 (3) | 0.94 ± 0.25 (6) | 0.99 ± 0.25 (4) |
| Tmax (h) | 1.03 (7) | 1.50 (3) | 1.04 (6) | 1.25 (4) |

¹Mean ± standard deviation (N) except for Tmax for which the median (N) is reported.
Source: Appendix VII.

1. A method for administering tolperisone, said method comprising:
administering a therapeutically effective dosage of tolperisone to a subject suffering from a condition responsive to treatment with tolperisone, wherein said subject is in a fed state.

2. The method of claim 1, wherein said administering takes place not more than approximately one hour following commencement of meal consumption by said subject.

3. The method of claim 2, wherein said administering takes place within approximately thirty minutes following commencement of meal consumption by said subject.

4. The method of claim 1, wherein the administering results in an elimination half-life of tolperisone in plasma longer than the elimination half-life achieved when tolperisone is administered to a subject in a fasted state.

5. The method of claim 4, wherein elimination half-life of tolperisone administered to the subject in the fed state is longer by at least 10% over that achieved when tolperisone is administered to a subject in a fasted state.

6. The method of claim 1, wherein the administering to said subject in the fed state results in enhanced bioavailability of tolperisone relative to that achieved when tolperisone is administered to a subject in a fasted state.

7. The method of claim 6, wherein the bioavailability of tolperisone in the subject is the fed state is at least 10% over that achieved when tolperisone is administered to a subject in a fasted state.

9. The method of claim 1, wherein the condition responsive to treatment with tolperisone is a condition selected from spasticity, muscle spasm, dysmenorrhea, climacteric complaints, lockjaw, neurolatyrism, deteriorated muscle tone, neuropathic pain, or pain associated with various nervous system disorders.

10. The method of claim 1, wherein the tolperisone is administered orally.

11. The method of claim 10, wherein the tolperisone is in a tablet, syrup, suspension, or a capsule.

12. The method of claim 1, wherein the therapeutically effective dosage of tolperisone ranges from approximately 50 milligrams to 1800 milligrams daily.

13. The method of claim 12, wherein the therapeutically effective dosage of tolperisone ranges from approximately 75 milligrams to 1500 milligrams daily.

14. The method of claim 1, wherein the tolperisone is in a form selected from the group consisting of a racemate, a chiral mixture, the R(−) enantiomer, or the S(+) enantiomer, and is optionally combined with a pharmaceutically-acceptable excipient.

15. A kit comprising:
tolperisone in packaged form, and
instructions for orally administering tolperisone to a subject within approximately 1 hour of meal commencement by the subject.

16. The kit of claim 15, comprising instructions for administering tolperisone within approximately 30 minutes of meal commencement.

17. The kit of claim 15, wherein said tolperisone is in a tablet, syrup, suspension, or capsule.
